(19) United States
(12) Patent Application Publication
(10) Pub. No.: US 2013/0149667 A1
(43) Pub. Date: Jun. 13, 2013

(54) MULTIPHASE TISSUE COMPLEX SCAFFOLDS

(71) Applicant: The Trustees of Columbia University in the City of New York, New York, NY (US)

(72) Inventors: Helen H. Lu, New York, NY (US); Nancy May Lee, New York, NY (US)

(73) Assignee: The Trustees of Columbia University in the City of New York, New York, NY (US)

(21) Appl. No.: 13/713,649

(22) Filed: Dec. 13, 2012

Related U.S. Application Data
(60) Provisional application No. 61/630,495, filed on Dec. 13, 2011.

(51) Int. Cl. A61K 6/087; A61K 6/04; A61K 6/033; A61C 8/00; A61C 5/00; A61K 6/02

(52) U.S. Cl. CPC A61K 6/087; A61C 5/00; A61K 6/0205; A61K 6/033; A61C 8/0012; A61K 6/04
USPC 433/173; 433/226; 433/228.1; 29/896.1

(57) ABSTRACT

Multiphase tissue engineered tissue complex scaffolds and methods for their use are provided.

### INTRODUCTION

This patent application claims the benefit of priority from U.S. Patent Application Ser. No. 61/630,495, filed Dec. 13, 2011, teachings of which are herein incorporated by reference in their entirety.

### STATEMENT REGARDING FEDERALLY SPONSORED RESEARCH OR DEVELOPMENT

This invention was made with government support under R01-AR055280-01 awarded by National Institutes of Health. The government has certain rights in the invention.

### FIELD

The disclosed subject matter relates to multiphase tissue complex scaffolds and methods of production and uses thereof.

### BACKGROUND

Twenty-five percent of adults 65 and older have lost all their teeth. The loss of the periodontal ligament (PDL) due to periodontal disease is a common cause of tooth loss.

Current treatments include open flap debridement, guided tissue regeneration involving a barrier membrane to prevent epithelial down-growth maintaining space for periodontal regeneration and bone graft with either an allograft or autograft.

Injectable hydrogels and bioscaffolds of microspheres have also been disclosed for use in periodontal ligament repair.

### SUMMARY

An aspect of the disclosed subject matter relates to a multiphase tissue engineered scaffold comprising a non-mineralized ligament phase with a folded, accordion-like structure and one or more mineralized phases adjacent to the non-mineralized ligament phase. In one embodiment, the multiphase tissue engineering scaffold is used in tissue complex regeneration and/or repair. In one embodiment, the multiphase tissue engineering scaffold is used in periodontium tissue complex regeneration and/or repair.

Another aspect of the disclosed subject matter relates to a periodontium tissue complex scaffold comprising a first mineralized phase for attachment of the scaffold to alveolar bone, a non-mineralized ligament phase adjacent to the first mineralized phase, and a second mineralized phase adjacent to the ligament phase for attachment of the scaffold to cementum. In one embodiment, the non-mineralized ligament phase has a folded, accordion-like structure.

Another aspect of the disclosed subject matter relates to a method for producing a multiphase tissue engineered scaffold. In one embodiment, the method comprises soaking one or more regions of a polymer nanofiber tissue engineered scaffold in one or more salt solutions to produce a tissue engineered scaffold with one or more mineralized phases and a ligament phase. In another embodiment, the method comprises electrospinning of a mineralized phase adjacent to a non-mineralized ligament phase. In this embodiment, the method may further comprise electrospinning a second mineralized phase so that the non-mineralized ligament phase is flanked between two mineralized phases.

Another aspect of the disclosed subject matter relates to a method for repairing or regenerating tissue complexes comprising implanting a multiphase tissue engineered scaffold disclosed herein adjacent to or near an injured or damaged tissue complex. In one embodiment the damaged tissue complex is the periodontium. In this embodiment, the method is used to inhibit tooth loosening in a subject. In this embodiment, a multiphase periodontal tissue engineered scaffold is implanted adjacent to a tooth of the subject.

Another aspect of the present invention relates to a method for biological fixation of an implant such as a dental implant with the multiphase tissue scaffold disclosed herein.

Another aspect of the disclosed subject matter relates to a method for promoting tissue complex regeneration. The method comprises seeding ligament-derived cells or cells capable of differentiating into ligament-like cells on a multiphase tissue engineered scaffold. In one embodiment, the seeded cells are periodontal ligament (PDL) derived cells or cells capable of differentiating into PDL-like cells and the tissue complex regenerated is the periodontium tissue complex.

Yet another aspect of the disclosed subject matter relates to a method for producing a tissue engineered ligament graft. The method comprises seeding ligament-derived cells or cells capable of differentiating into ligament-like cells on a multiphase tissue engineering scaffold. In one embodiment, the seeded cells are PDL derived cells or cells capable of differentiating into PDL-like cells and the tissue engineered ligament graft is a periodontal tissue engineered ligament graft.

### BRIEF DESCRIPTION OF THE FIGURES

FIGS. 1A-C provide schematics of an embodiment of a multiphase tissue engineered scaffold of this disclosure and its use in periodontium tissue complex regeneration. In this embodiment, as depicted in FIG. 1A, a non-mineralized ligament phase is flanked by mineralized regions. FIG. 1B provides a closer view of the folded, accordion like structure of the non-mineralized ligament phase of this tissue scaffold embodiment. FIG. 1C provides a schematic of implantation of this tissue scaffold at the defect site.

FIG. 2 provides another schematic of an embodiment of a multiphase tissue engineered scaffold of this disclosure comprising a non-mineralized ligament phase flanked by mineralized regions (FIG. 2A) and scanning electron microscopy (SEM) images of the mineralized regions (FIGS. 2B and 2D) and non-mineralized phase (FIG. 2C). In this scaffold embodiment of electrospun nanofibers, mineralized regions were formed through soaking in a simulated body fluid (SBF) solution.

FIG. 3 provides another schematic of an embodiment of a multiphase tissue engineered scaffold of this disclosure comprising a non-mineralized ligament phase flanked by mineralized regions (FIG. 3A) and SEM micrographs of the mineralized regions (FIGS. 3B and 3D) and non-mineralized phase (FIG. 3C). In this scaffold embodiment of electrospun nanofibers, mineralized regions were formed from electrospinning hydroxyapatite onto the scaffold.

FIG. 4 is an SEM micrograph of the interface between mineralized and non-mineralized regions of the multiphase tissue engineered scaffold of FIG. 3.

FIG. 5 is a schematic depicting integration of a mineralized region of an embodiment of a scaffold of this disclosure prepared either by soaking the electrospun nanofibers in a simulated body fluid (SBF) solution or electrospinning hydroxyapatite onto the scaffold with a titanium dental implant.

FIGS. 6A and 6B show alternative scaffold designs of this disclosure produced either by electrospinning non-mineralized and mineralized scaffolds separately and sandwiching the non-mineralized scaffold between mineralized scaffolds or by electrospinning the entire scaffold of a non-mineralized ligament phase flanked by mineralized regions in the same fabrication process. In these embodiments, the non-mineralized scaffold comprises electrospun nanofibers of polycaprolactone (PCL) and the mineralized scaffolds comprise electrospun nanofibers of polycaprolactone (PCL) and hydroxyapatite (HA). FIG. 6A shows an embodiment wherein the nanofibers are unaligned. FIG. 6B shows an embodiment wherein the nanofibers are aligned.

FIG. 7 provides schematics of application of the alternative design of FIG. 6 through implantation at the defect site (FIG. 7A) or integration with an implant (FIG. 7B).

FIG. 8 provides a comparison of PDL cell growth on PLGA aligned nanofiber scaffolds versus PCL aligned nanofiber scaffolds on days 1, 7, 14 and 28 of culture.

FIG. 9 provides a comparison of ALP activity (FIG. 9A) and collagen deposition (FIG. 9B) of PDL cells grown on PLGA aligned nanofiber scaffolds versus PCL aligned nanofiber scaffolds on days 1, 7, 14 and 28 of culture.

FIG. 10 provides SEM micrographs of the non-mineralized, mineralized and transition phases of a PCL nanofiber tissue scaffold and shows PDL cell attachment and viability as determined through live/dead staining on day 1 to all three phases.

FIG. 11 provides of a comparison of PDL cell growth on a non-mineralized PLGA aligned nanofiber scaffold versus a mineralized PLGA-HA aligned nanofiber scaffold on Days 1, 7, 14 and 28 of culture.

FIG. 12 provides a comparison of ALP activity (FIG. 12A) and collagen deposition (FIG. 12B) of PDL cells grown on non-mineralized PLGA aligned nanofiber scaffolds versus mineralized PLGA-HA aligned nanofiber scaffolds on days 1, 7, 14 and 28 of culture and day 28 of culture, respectively.

FIG. 13 provides a comparison of PDL cell growth on PCL aligned nanofiber scaffolds versus PCL unaligned nanofiber scaffolds on days 1, 7, 14 and 28 of culture.

FIG. 14 provides a comparison of ALP activity (FIG. 14A) and collagen deposition (FIG. 14B) of PDL cells grown on PCL aligned nanofiber scaffolds versus PCL unaligned nanofiber scaffolds on days 1, 7, 14 and 28 of culture.

### DETAILED DESCRIPTION

### Definitions

In order to facilitate an understanding of the material which follows, one may refer to Freshney, R. Ian. Culture of Animal Cells—A Manual of Basic Technique (New York: Wiley-Liss, 2000) for certain frequently occurring methodologies and/or terms which are described therein.

Unless defined otherwise, all technical and scientific terms used herein have the meaning commonly understood by a person skilled in the art to which this invention belongs. However, except as otherwise expressly provided herein, each of the following terms, as used in this application, shall have the meaning set forth below.

As used herein, “active agent” shall mean a component incorporated into the multiphase tissue scaffold, which when released over time, supports alignment, proliferation and matrix deposition of a selected ligament cell. Examples include, but are in no way limited to growth factors such as transforming growth factor-beta 3(TGF-β3), growth/differentiation factor-5 (gdf-5), bone morphogenetic protein (BMP) 1 through 14, fibroblast growth factor (FGF) and basic fibroblast growth factor (bGF). A single active agent or a combination of active agents may be incorporated into the tissue engineering scaffolds of this application. By “active agent” it is also meant to include an active pharmaceutical ingredient such as, but not limited to, an anti-inflammatory, an antibiotic or a pain medicament added to the multiphase tissue scaffold to enhance treatment and/or healing of the subject upon implantation.

As used herein, “aligned fibers” shall mean groups of fibers which are oriented along the same directional axis. Examples of aligned fibers include, but are not limited to, groups of parallel fibers.

As used herein, a “biocompatible” material is a synthetic or natural material used to replace part of a living system or to function in intimate contact with living tissue. Biocompatible materials are intended to interface with biological systems to evaluate, treat, augment or replace any tissue, organ or of the body. The biocompatible material has the ability to perform with an appropriate host response in a specific application and does not have toxic or injurious effects on biological systems. Nonlimiting examples of biocompatible materials include a biocompatible ceramic, a biocompatible polymer or a biocompatible hydrogel.

As used herein, “biodegradable” means that the material, once implanted into a host, will begin to degrade.

As used herein, “biomimetic” shall mean a resemblance of a synthesized material to a substance that occurs naturally in a human body and which is not substantially rejected by (e.g., does not cause an unacceptable adverse reaction in) the human body. When used in connection with the tissue scaffolds, biomimetic means that the scaffold is substantially biologically inert (i.e., will not cause an unacceptable immune response/rejection) and is designed to resemble a structure (e.g., soft tissue anatomy) that occurs naturally in a mammalian, e.g., human, body and that promotes healing when implanted into the body.

As used herein, “nanofiber” shall mean a fiber with a diameter no more than 1000 nanometers.

In one embodiment, the nanofibers are comprised of a polymer that is electrospun into a fiber. The nanofibers of the scaffold are oriented in such a way (i.e., aligned or unaligned) so as to mimic the natural architecture of the soft tissue to be repaired. Moreover, the nanofibers and the subsequently formed nanofiber scaffolds are controlled with respect to their physical properties, such as for example, fiber diameter, pore diameter, and porosity so that the mechanical properties of the nanofibers and nanofiber scaffolds are similar to the native tissue to be repaired, augmented or replaced.

As used herein, “polymer” means a chemical compound or mixture of compounds formed by polymerization and including repeating structural units. Polymers may be constructed in multiple forms and compositions or combinations of compositions and may be degradable or nondegradable.

As used herein, “stem cell” means any unspecialized cell that has the potential to develop into many different cell types in the body, such as ligament cells, and in particular periodontal ligament cells. Nonlimiting examples of “stem cells” include mesenchymal stem cells, embryonic stem cells and induced pluripotent cells.

As used herein, “synthetic” shall mean that the material is not of a human or animal origin.

As used herein, “tissue complex” is meant to include any soft and hard tissues connected by a ligament, as well as the ligament, damage to which can be repaired and/or the tissue complex regenerated using the multiphase tissue engineered scaffolds of this disclosure. Examples include, but are in no way limited to, the periodontium tissue complex consisting of the alveolar bone, the periodontal ligament (PDL), and the cementum and the medial collateral ligament (MCL) to bone insertion.

As used herein, all numerical ranges provided are intended to expressly include at least the endpoints and all numbers that fall between the endpoints of ranges.

The following embodiments are provided to further illustrate the methods of tissue scaffold production of this application. These embodiments are illustrative only and are not intended to limit the scope of this application in any way.

### Embodiments

The disclosed subject matter relates to multiphase tissue scaffolds, methods for producing these multiphase tissue scaffolds and methods for their use in promoting tissue complex regeneration.

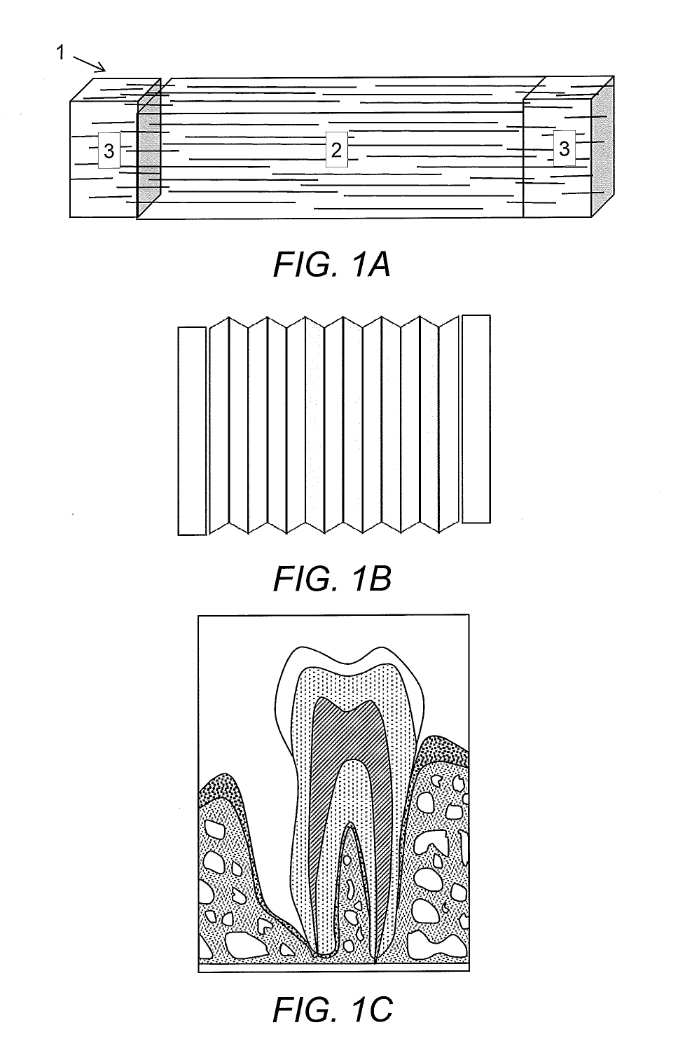

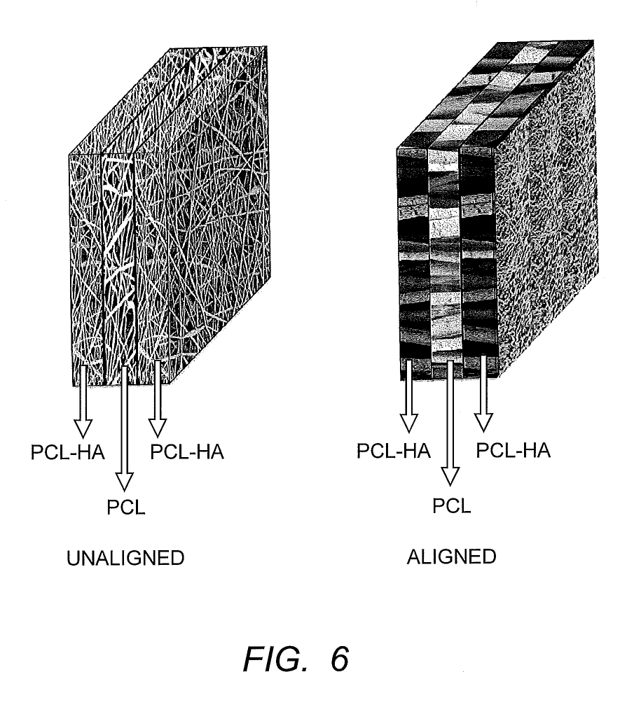

The multiphase tissue scaffolds of this disclosure comprise a non-mineralized ligament phase and one or more mineralized phases adjacent to the non-mineralized ligament phase. A number of nonlimiting embodiments of multiphase tissue scaffolds of this disclosure with a non-mineralized ligament phase flanked by mineralized regions or phases are depicted in FIGS. 1 through 7. The depicted embodiments in FIGS. 1 through 7 of a non-mineralized ligament phase flanked by mineralized regions or phases provide a mimetic of the soft-to-hard tissue interfaces connected via ligaments and facilitate the integration and regeneration of ligament to mineralized cementum and/or bone via the tissue scaffolds of this disclosure.

The non-mineralized ligament phase 2 of the multiphase scaffold 1 is comprised of biocompatible and/or biodegradable polymeric or copolymeric nanofibers. It is expected that any biocompatible and/or biodegradable or nondegradable polymeric or copolymeric nanofibers or ECM matrices can be used in the non-mineralized ligament phase. In one embodiment, the nanofibers comprise poly(lactic-co-glycolic acid (PLGA), poly(lactide) (PLA) or poly(glycolide)(PGA). In another embodiment, the nanofibers comprise polycaprolactone (PCL). In another embodiment, the nanofibers comprise a blend of PLGA, PLA and/or PGA and PCL. However, as will be understood by the skilled artisan upon reading this disclosure, alternative polymers or copolymers with similar functional and/or structural characteristics can also be used.

In one embodiment, nanofibers of the non-mineralized ligament phase of the scaffold are aligned. In another embodiment the nanofibers of the non-mineralized ligament phase of the scaffold are unaligned.

In one embodiment, the non-mineralized ligament phase is folded into an accordion-like structure as depicted in FIG. 1B.

In one embodiment, length, width and/or size of the non-mineralized phase is selected to mimic the native ligament of a selected tissue complex. For example, in the periodontium tissue complex, the native PDL is 0.15 to 0.38 mm. Accordingly, in embodiments of this disclosure used for periodontium tissue complex regeneration, length of non-mineralized phase can range from 0.15 to 0.38 mm. Further, the number and depth of the folds of the accordion-like structure can be adjusted and/or customized to accommodate to the depth and/or size of a defect in individual patients.

The mineralized phase 3 of the multiphase scaffold 1 also comprises biocompatible and/or biodegradable polymeric or copolymeric nanofibers. It is expected that any biocompatible and/or biodegradable polymeric or copolymeric nanofibers or ECM matrices can be used in the mineralized phase. In one embodiment, the nanofibers comprise poly(lactic-co-glycolic acid (PLGA), poly(lactide) (PLA) or poly(glycolide)(PGA). In another embodiment, the nanofibers comprise polycaprolactone (PCL). In another embodiment, the nanofibers comprise a blend of PLGA, PLA and/or PGA and PCL. However, as will be understood by the skilled artisan upon reading this disclosure, alternative polymers or copolymers with similar functional and/or structural characteristics can also be used.

In one embodiment, nanofibers of the mineralized phase of the scaffold are aligned. In another embodiment the nanofibers of the mineralized phase of the scaffold are unaligned.

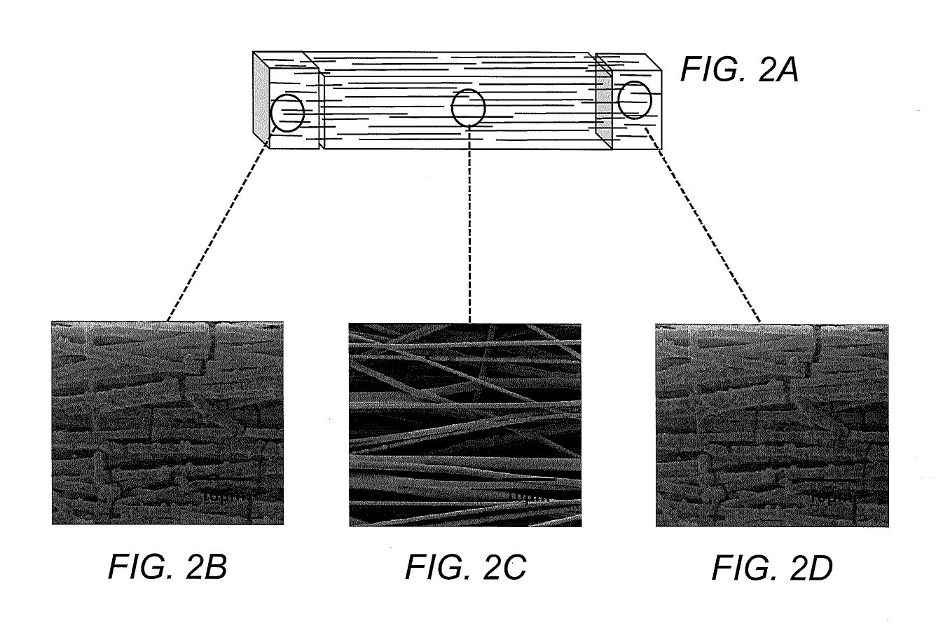

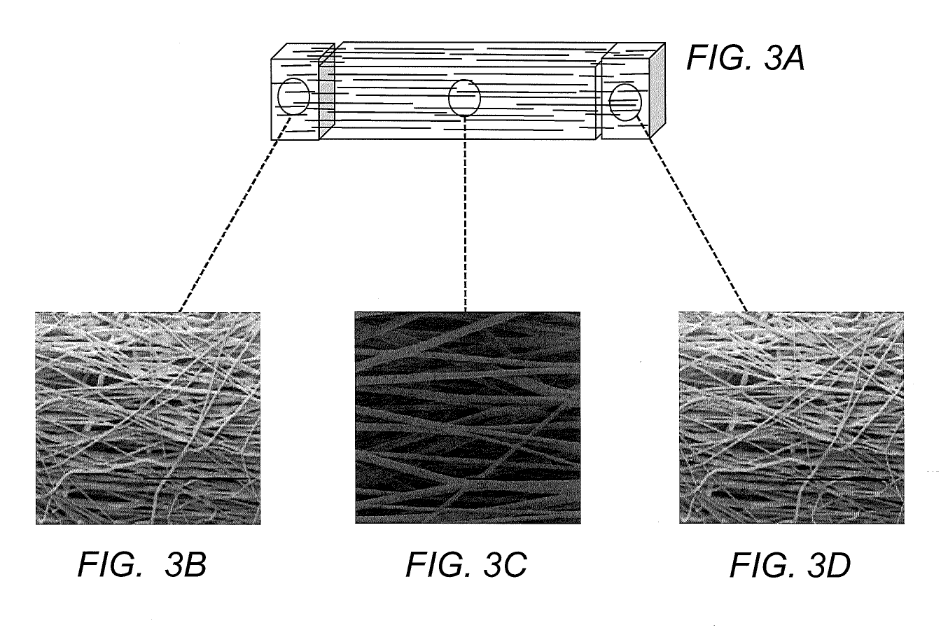

Various methods for mineralizing polymeric or copolymeric nanofibers to produce the one or more mineralized phases of the multiphase tissue scaffolds of this disclosure are available. For example, in one embodiment, as depicted in FIG. 3, the mineralized phases are produced by direct electrospinning of a polymer solution containing a ceramic. While examples herein relate to hydroxyapatite, as will be understood by the skilled artisan upon reading this disclosure, any calcium phosphate can be used. In another embodiment, as depicted in FIG. 2, the mineralized phases are produced by soaking the nanofiber scaffold in a series of concentration salt solutions such as Simulated Body Fluid or SBF as described, for example, by Habibovic et al. (J. Amer. Ceramic Soc. 2002 85(3):517-522) and Lu et al. (J. Biomed. Mater. & Res. 2000 51:80-87). As shown in FIG. 6, the multiphase tissue scaffolds of this disclosure can also be produced by electrospinning non-mineralized and mineralized scaffold separately and then sandwiching the non-mineralized scaffold between mineralized scaffolds or by electrospinning the entire scaffold of a non-mineralized ligament phase flanked by mineralized regions in the same fabrication process.

Length, width and size of the mineralized phase or phases can be adjusted depending upon the defect site and/or tissue complex to be regenerated with the tissue scaffold.

In one embodiment, the multiphase tissue scaffold of this disclosure may further comprise an active agent in the non-mineralized phase and/or the one or more mineralized phases. Examples of active agents include, but are in no way limited to, growth factors, cytokines and cells, which when incorporated into the multiphase tissue scaffold, supports alignment, proliferation and matrix deposition of a selected ligament cell, and active pharmaceutical agents such as, but not limited to, anti-inflammatory agents, antibiotics or pain medicines which may enhance treatment and or tissue complex healing of the subject upon implantation of the multiphase tissue scaffold.

In one embodiment, the scaffolds of this disclosure may further comprise ligament-derived cells or cells capable of differentiating into ligament-like cells such as, but not limited to, stem cells. In one embodiment, the cells are human ligament-derived cells.

By “ligament-like cells” is it meant to include any cell which expresses ligament markers and/or supports formation of a ligament-like tissue.

In one nonlimiting embodiment, the multiphase tissue engineered scaffolds are used to regenerate or repair the periodontium tissue complex consisting of the alveolar bone, the periodontal ligament (PDL), and the cementum periodontal ligament. The PDL is a soft, highly vascularized, connective tissue 0.15-0.38 mm in width which transmits forces to be distributed and adsorbed by the alveolar bone and participated in tooth mobility. In one embodiment, the periodontium tissue complex scaffold comprises a first mineralized phase for attachment of the scaffold to alveolar bone, a non-mineralized ligament phase adjacent to the first mineralized; and a second mineralized phase adjacent to the ligament phase for attachment of the scaffold to cementum.

In one embodiment, the periodontium tissue complex scaffold further comprises PDL-derived cells or cells capable of differentiating into PDL-like cells. In one embodiment, the cells are human PDL-derived cells. In one embodiment, the cells are stem cells. In these embodiments, the polymer nanofiber architecture and/or blend of polymers may be selected for optimal periodontium tissue complex regeneration in accordance with teachings herein.

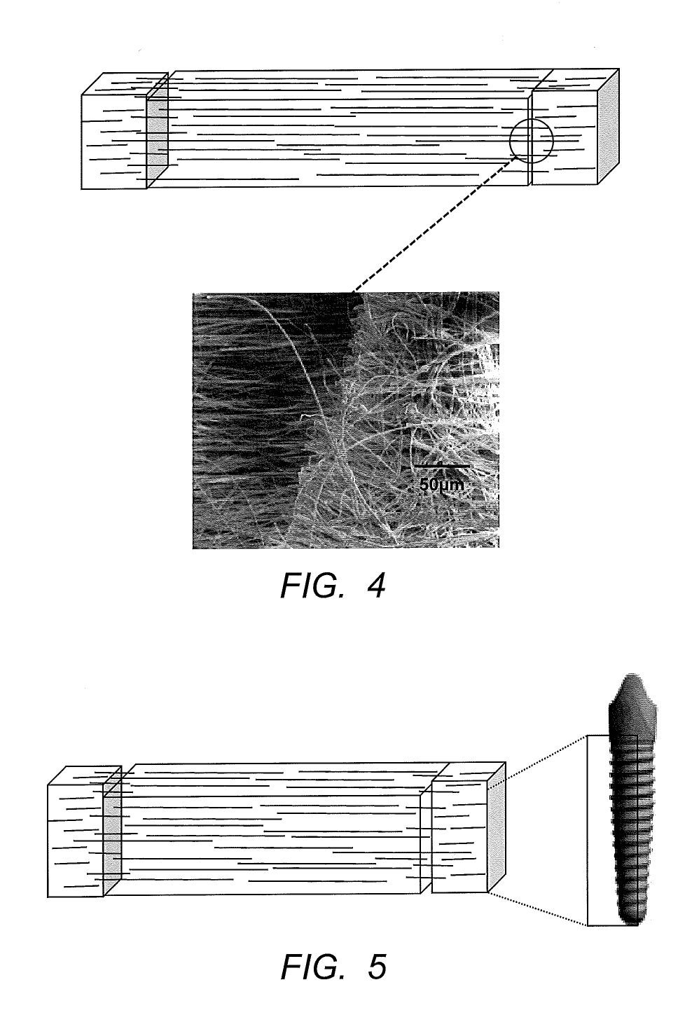

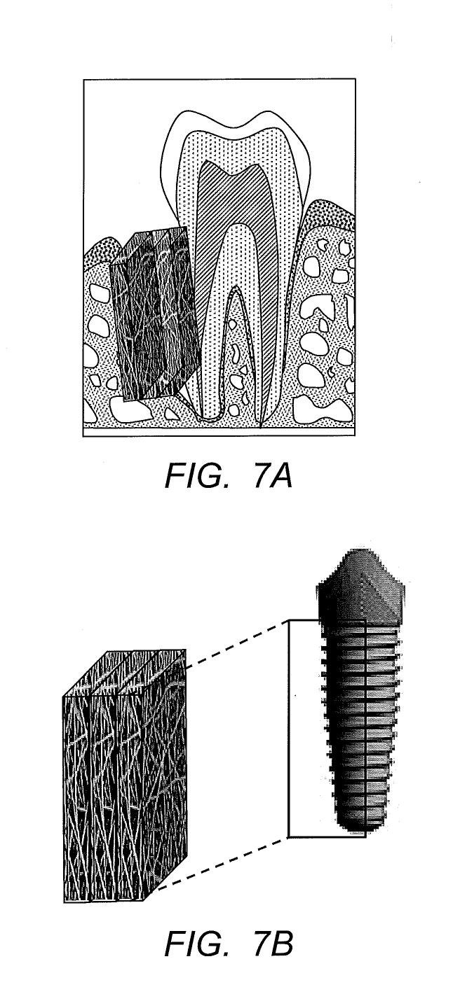

Implantation of embodiments of a periodontium tissue complex scaffold of this disclosure at a defect site are depicted in FIG. 1C and FIG. 7A. Integration of the mineralized region of periodontium tissue complex scaffolds of this disclosure prepared with a titanium dental implant are depicted in FIG. 4 and FIG. 7B.

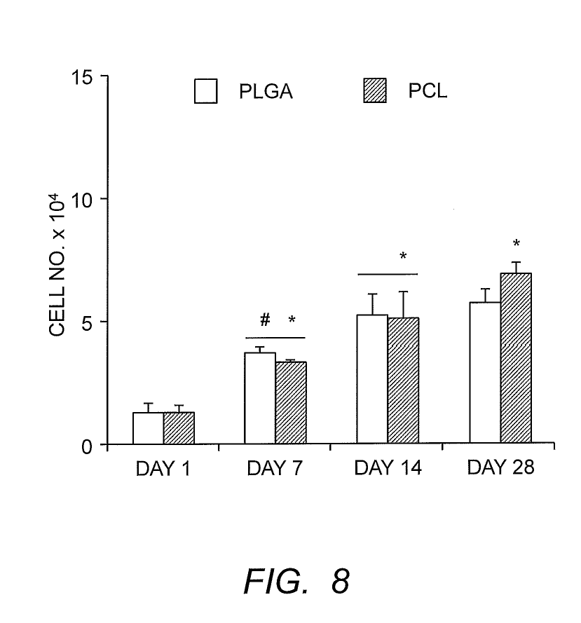

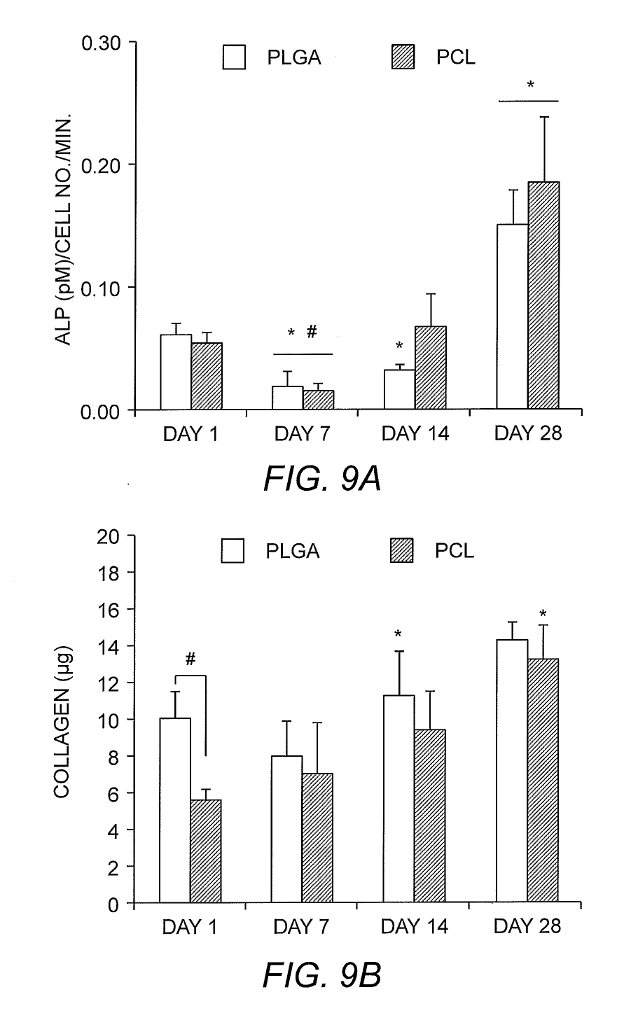

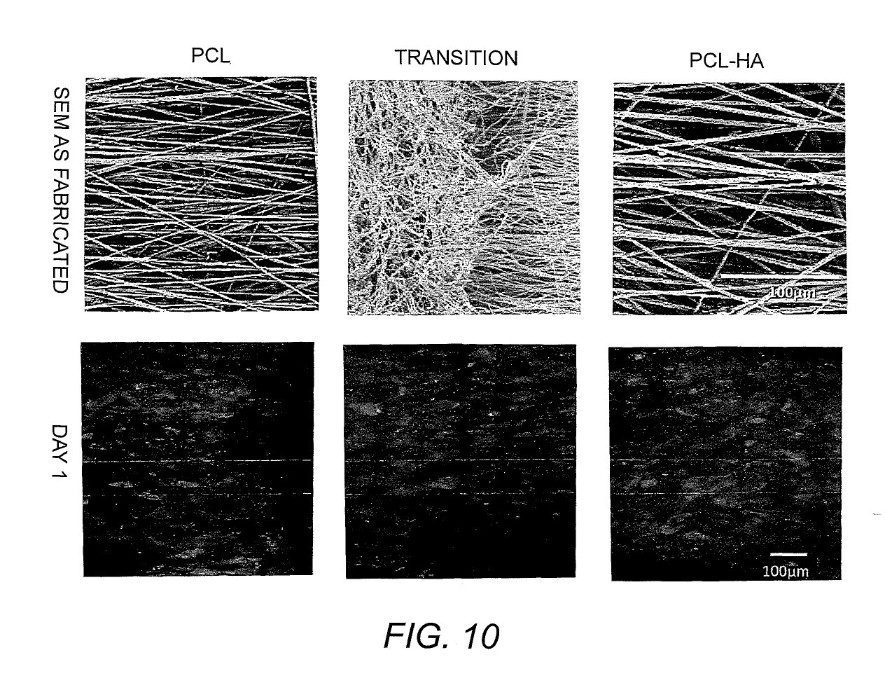

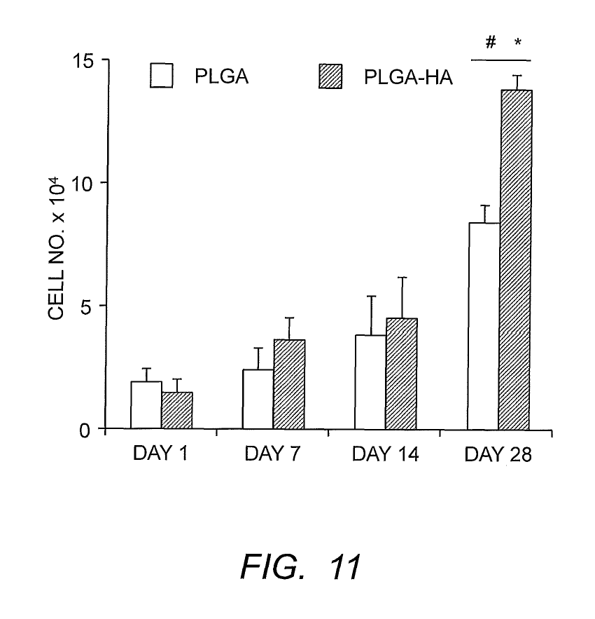

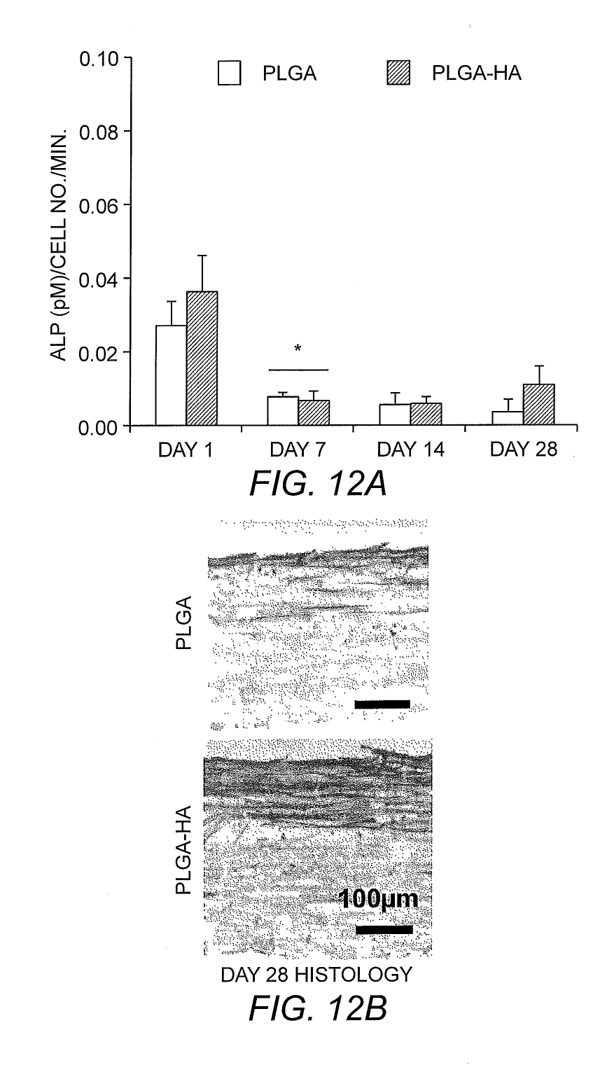

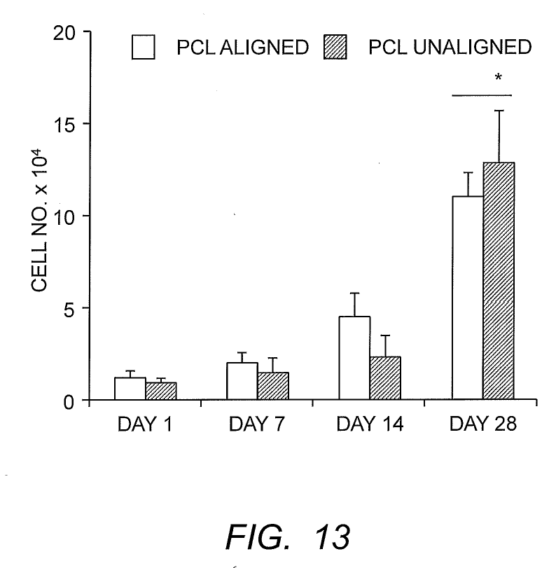

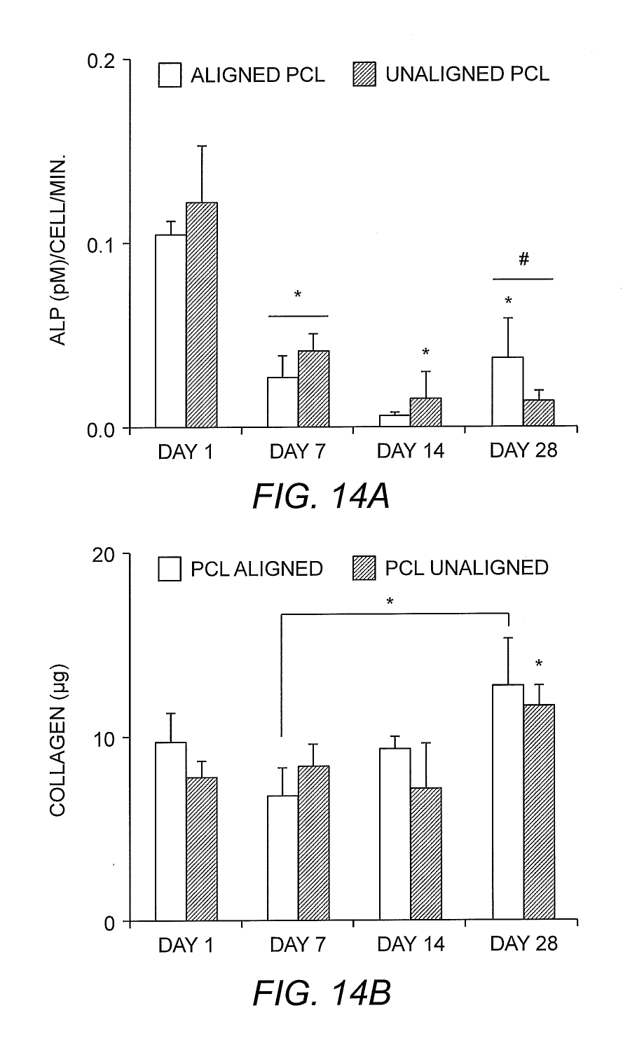

Multiphase tissue scaffolds of this disclosure comprising PCL nanofibers and multiphase tissue scaffolds of this disclosure comprising PCL nanofibers, each seeded with PDL cells, were prepared. Experiments were performed comparing cell viability, alignment, proliferation, alkaline phosphatase (ALP activity) and collagen deposition on these different scaffolds. Results are depicted in FIGS. 8 through 14. As shown in FIG. 8, cell growth was similar on PLGA and PCL aligned nanofiber scaffolds on days 1, 7, 14 and 28. Also similar on the aligned PLGA and PCL nanofiber scaffolds were ALP activity (see FIG. 9A) and collagen deposition (see FIG. 9B). Cells attached and were viable on the non-mineralized ligament phase, and mineralized phase and the transition region of the two phases after one day of culture on the aligned PCL nanofiber scaffold (see FIG. 10). However, greater cell proliferation was observed on the mineralized phase of the aligned PLGA nanofiber scaffolds at Day 28 (see FIG. 11). Further, while similar ALP activity was observed, greater collagen deposition was observed on the mineralized phase of the aligned PLGA nanofiber scaffolds at Day 28 (see FIG. 12). No difference was observed in cell proliferation (see FIG. 13) or collagen deposition (see FIG. 14B) between aligned PCL nanofiber scaffolds and unaligned PCL nanofiber scaffolds. However, ALP activity was enhanced on aligned PCL scaffolds (see FIG. 14A).

Experiments were also performed to determine gene expression of the PDL cells. All scaffolds supported the expression of type I collagen, fibromodulin, and bone sialoprotein (BSP). Further, significant upregulation of periostin, a PDL specific marker, was observed in PDL cells grown on the PCL scaffolds.

Accordingly, these experiments are indicative of the multiphase tissue scaffolds of this disclosure being useful in tissue complex regeneration and/or repair, and in particular periodontium tissue complex regeneration and repair. Tissue engineered scaffolds of this disclosure are useful in regenerating the cementum-periodontal ligament bone complex and thus provide a useful means for preventing or inhibiting tooth loss and augmenting dental implants.

The disclosed subject matter is further illustrated by the following nonlimiting examples.

### EXAMPLES

### Example 1

### Scaffold Fabrication and Cell Culture

Aligned PLGA (85:15, Lakeshore) or PCL (Sigma) nanofiber scaffolds were fabricated by electrospinning. The PLGA polymer solution used consisted of 54% w/v in DMF (Sigma) and ethanol. The PCL polymer solution used consisted of 16% w/v in DMF and DCM (2:3). Polymer solutions were electrospun at 1.0 mL/hr at 8-10 kV and collected on a rotating mandrel.

Human PDL cells were derived from explant culture of healthy PDL after tooth extraction. Cells at passage 4 were seeded at 30,000 cells/cm² on scaffolds and cultured in DMEM+10% FBS with ascorbic acid supplementation.

### Example 2

### End-Point Analyses

Samples were analyzed after 1, 7, 14, and 28 days of culture.

Cell viability, attachment, and morphology (n=3) were evaluated using Live/Dead assay (Molecular Probes) with cell alignment determined using custom software as described by Costa et al. (Tissue Eng, 2003; 9(4), 567-77). Cell proliferation (n=6) was measured by DNA quantitation (PicoGreen®, Molecular Probes). Alkaline phosphatase activity was determined (n=6) using an enzymatic assay. Collagen deposition was quantified (n=6) with a modified hydroxyproline assay as described by Reddy et al. (Clin Biochem, 1996; 29(3), 225-99).

Collagen I, bone sialoprotein, fibromodulin, and periostin expression were evaluated (n=4) by RT-PCR with GAPDH expression serving as a normalization factor.

Two-way ANOVA was performed and Tukey-Kramer test was used for all pair-wise comparisons with statistical significance determined at p<0.05.

This disclosure should not be construed as limiting the invention in any way. One of skill in the art will appreciate that numerous modifications, combinations, rearrangements, etc. are possible without exceeding the scope of the invention. While this invention has been described with an emphasis upon various embodiments, it will be understood by those of ordinary skill in the art that variations of the disclosed embodiments can be used, and that it is intended that the invention can be practiced otherwise than as specifically described herein.

What is claimed is:

1. A multiphase tissue engineered scaffold comprising:
a non-mineralized ligament phase with a folded, accordion-like structure; and
one or more mineralized phases adjacent to the non-mineralized ligament phase.

2. The multiphase tissue engineered scaffold of claim 1 comprising:
a non-mineralized ligament phase with a folded, accordion-like structure; and
first and second mineralized phases adjacent to the non-mineralized ligament phase.

3. The multiphase tissue engineered scaffold of claim 1 wherein the non-mineralized ligament phase comprises polymer nanofibers.

4. The multiphase tissue engineered scaffold of claim 3 wherein the polymer nanofibers have a selected architecture and/or comprise a blend of polymers optimal for periodontium tissue complex regeneration.

5. The multiphase tissue engineered scaffold of claim 3 wherein the polymer nanofibers comprise PLGA, PLA or PGA.

6. The multiphase tissue engineered scaffold of claim 3 wherein the polymer nanofibers comprise polycaprolactone (PCL).

7. The multiphase tissue engineered scaffold of claim 3 wherein the polymer nanofibers comprise a blend of PLGA, PLA and/or PGA and PCL.

8. The multiphase tissue engineered scaffold of claim 3 wherein the polymer nanofibers are aligned.

9. The multiphase tissue engineered scaffold of claim 3 wherein the polymer nanofibers are unaligned.

10. The multiphase tissue engineered scaffold of claim 1 wherein the one or more mineralized phases comprise polymer nanofibers and a ceramic.

11. The multiphase tissue engineered scaffold of claim 1 wherein the one or more mineralized phases comprise polymer nanofibers and hydroxyapatite or a calcium phosphate.

12. The multiphase tissue engineered scaffold of claim 11 wherein the polymer nanofibers comprise PLGA, PLA or PGA.

13. The multiphase tissue engineered scaffold of claim 11 wherein the polymer nanofibers comprise polycaprolactone (PCL).

14. The multiphase tissue engineered scaffold of claim 11 wherein the polymer nanofibers comprise a blend of PLGA, PLA and/or PGA and PCL.

15. The multiphase tissue engineered scaffold of claim 11 wherein the polymer nanofibers are aligned.

16. The multiphase tissue engineered scaffold of claim 11 wherein the polymer nanofibers are unaligned.

17. The multiphase tissue engineered scaffold of claim 11 wherein the one or more mineralized phases are produced by electrospinning a ceramic onto the polymer nanofibers.

18. The multiphase tissue engineered scaffold of claim 11 wherein the one or more mineralized phases are produced by electrospinning hydroxyapatite or a calcium phosphate onto the polymer nanofibers.

19. The multiphase tissue engineered scaffold of claim 11 wherein the one or more mineralized phases are produced by soaking a region of the scaffold in one or more concentrated salt solutions.

20. The multiphase tissue engineered scaffold of claim 1 further comprising an active agent in the non-mineralized ligament phase and/or the one or more mineralized phases.

21. The multiphase tissue engineered scaffold of claim 20 wherein the active agent is an antibiotic.

22. The multiphase tissue engineered scaffold of claim 1 wherein number and/or depth of folds in the accordion-like structure of the non-mineralized phase are customized to accommodate to depth and/or size of a defect in a patient.

23. The multiphase tissue engineered scaffold of claim 4 seeded with PDL-derived cells or cells capable of differentiating into PDL-like cells.

24. A method for producing a multiphase tissue engineered ligament graft, said method comprising soaking one or more regions of a polymer nanofiber tissue engineered scaffold in one or more salt solutions to produce a tissue engineered scaffold with one or more mineralized phases and a non-mineralized ligament phase.

25. The method of claim 24 wherein the non-mineralized ligament phase has a folded, accordion-like structure.

26. A method for inhibit tooth loosening in a subject comprising implanting the multiphase tissue engineered scaffold of claim 1 adjacent to a tooth of the subject.

27. A method for biologically fixing an implant in a subject, said method comprising interfacing the multiphase tissue engineered scaffold of claim 1 with an implant and implanting the interfaced scaffold and implant in a subject.

28. The method of claim 27 wherein the implant is a dental implant.

29. A periodontium tissue complex scaffold comprising:
a first mineralized phase for attachment of the scaffold to alveolar bone;
a non-mineralized ligament phase adjacent to said first mineralized phase; and
a second mineralized phase adjacent to said ligament phase for attachment of the scaffold to cementum.

30. The periodontium tissue complex scaffold of claim 29 wherein said non-mineralized ligament phase has a folded, accordion-like structure.

31. The periodontium tissue complex scaffold of claim 30 wherein number and/or depth of folds in the accordion-like structure of the non-mineralized phase are customized to accommodate to depth and/or size of a defect in a patient.

32. The periodontium tissue complex scaffold of claim 29 produced by electrospinning non-mineralized and mineralized scaffolds separately and sandwiching the non-mineralized scaffold between mineralized scaffolds to form the periodontium tissue complex scaffold.

33. The periodontium tissue complex scaffold of claim 29 produced by electrospinning a scaffold of a non-mineralized ligament phase flanked by mineralized regions in a single fabrication process.

34. A method for inhibit tooth loosening in a subject comprising implanting the multiphase tissue engineered scaffold of claim 29 adjacent to a tooth of the subject.

35. A method for biologically fixing a dental implant in a subject, said method comprising interfacing the multiphase tissue engineered scaffold of claim 29 with the implant and implanting the interfaced scaffold and dental implant in the subject.
